SEQUENCE LISTING (9 sequences)

SEQ ID NO: 1 - RNA, 87 nt, HOMO SAPIENS
gacagugcagucacccauaaaguagaaagcacuacuaacagcacuggaggguguaguguuuccuacuuuauggaugaguguacugug

SEQ ID NO: 2 - RNA, 75 nt, Homo sapiens
ugcccuggcucaguuaucacagugcugaugcugucuauucuaaagguacaguacugugauaacugaaggauggca

SEQ ID NO: 3 - RNA, 78 nt, Homo sapiens
gccgcaggugcucugacgagguugcacuacugugcucugagaagcagugcaaugauauugucaaagcaucugggacca

SEQ ID NO: 4 - RNA, 68 nt, Homo sapiens
gaggcaaaguucugagacacuccgacucugaguaugauagaagucagugcacuacagaacuuugucuc

SEQ ID NO: 5 - RNA, 72 nt, Homo sapiens
ugucggguagcuuaucagacugauguugacuguugaaucucauggcaacaccagucgaugggcugucugaca

SEQ ID NO: 6 - RNA, 88 nt, Homo sapiens
aucucuuacacaggcugaccgauuucuccugguguucagagucuguuuuugucuagcaccauuugaaaucgguuaugauguaggggga

SEQ ID NO: 7 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 8 - RNA, 78 nt, Homo sapiens
cugaggagcagggcuuagcugcuugugagcaggguccacaccaagucguguucacaguggcuaaguuccgccccccag

SEQ ID NO: 9 - RNA, 83 nt, Homo sapiens
ccuuggaguaaaguagcagcacauaaugguuuguggauuuugaaaaggugcaggccauauugugcugccucaaaaauacaagg

(19) United States
(12) Patent Application Publication
(10) Pub. No.: US 2016/0032383 A1
(43) Pub. Date: Feb. 4, 2016

(54) PANEL OF microRNA BIOMARKERS IN HEALTHY AGING

(71) Applicant: ALBERT EINSTEIN COLLEGE OF MEDICINE OF YESHIVA UNIVERSITY, Bronx, NY (US)

(72) Inventor: Yousin SUH, New York, NY (US)

(73) Assignee: ALBERT EINSTEIN COLLEGE OF MEDICINE OF YESHIVA UNIVERSITY, Bronx, NY (US)

(21) Appl. No.: 14/773,396

(22) PCT Filed: Mar. 14, 2014
(86) PCT No.: PCT/US14/27113

§ 371 Date: Sep. 8, 2015

Related U.S. Application Data
(60) Provisional application No. 61/791,426, filed on Mar. 15, 2013.

(51) Int. Cl. C12Q 1/68; A61K 31/7105

(52) U.S. Cl. CPC C12Q 1/6883; A61K 31/7105; C12Q 2600/178

(57) ABSTRACT

Methods are provided for determining if a subject is likely to develop an age-related disease based on miRNA signatures. Related methods of treatment are also provided.

### CROSS-REFERENCE TO RELATED APPLICATIONS

This application claims benefit of U.S. Provisional Application No. 61/791,426, filed Mar. 15, 2013, the contents of which are hereby incorporated by reference.

### BACKGROUND OF THE INVENTION

Throughout this application various publications are referred to, including by number in parentheses. Full citations for these references may be found at the end of the specification. The disclosures of these publications, and of all patents, patent application publications and books referred to herein, are hereby incorporated by reference in their entirety into the subject application to more fully describe the art to which the subject invention pertains.

Since the introduction of high throughput technology to measure genome-wide gene expression levels, mounting evidence in model organisms indicates that aging is accompanied by enhanced gene expression variation (26,27) and a decline in gene co-expression network integrity (28,29). These results suggest that aging may affect major gene expression regulators leading to deregulation of many downstream targets, having a major impact on cell and tissue function, disease risk, and lifespan. Recently, miRNAs have emerged as critical regulators of gene expression and have been linked to longevity (30,31) and aging (32) in C. elegans.

MicroRNAs (miRNAs), first discovered in C. elegans (33), are small non-coding RNA species that post-transcriptionally regulate gene expression (34). Mature miRNAs, 18-25 bp in length, are transcribed as primary-miRNA (pri-miRNA) molecules containing a characteristic stem loop structure. This stem loop targets pri-miRNA for processing by a number of RNAses, namely Drosha and Dicer, which produce a short RNA duplex (34). From the duplex, one or both strands are incorporated into the RNA inducing silencing complex (RISC), resulting in an active miRNA. The active miRNA primarily target the 3′ UTR of a mRNA based on sequence homology (35). The nucleotides in the 2-7 position of the 5′ end of the mature miRNA comprise a “seed region.” Absolute homology in this region is required for miRNA to target a given mRNA (36). Once an mRNA is targeted by a miRNA, its gene expression is down-regulated due to induction of mRNA degradation or by blocking translation through conserved mechanisms (34,37). Since one miRNA can bind multiple mRNA targets, miRNAs can significantly alter gene regulatory networks. In-depth study and characterization of miRNA impact has elucidated their critical functions in development, homeostasis, and disorders including cardiovascular (38) and neurodegenerative disease (39). Thus far, 1048 human miRNA sequences have been identified through cloning, sequencing, or computational analysis (mirBase, release 16, 2010) (40,41) and in silico analysis predicts that they may regulate up to ⅓rd of the human genome (42).

Multiple miRNAs have been shown to regulate life span of C. elegans both positively and negatively (30,31,43) adding weight to the hypothesis that this gene class may contribute to robustness required for maintenance of healthy life span (44). For example, reducing the activity of miRNA, lin-4, shortened life span and accelerated tissue aging, whereas overexpression of lin-4 extended life span by suppressing the target gene, lin-14 (30). Furthermore, expression patterns of these lifespan-modulating miRNAs can be a predictor of lifespan in C. elegans (43); they control gene expression involved in major conserved pathways that impact life span, such as the insulin/IGF-1 signaling pathway (30,31,43). Recently, miRNAs were shown to mediate the longevity phenotype in mammals, namely, Ames dwarf mice (45), implicating a role in mammalian longevity. Since a significant number of miRNAs are evolutionarily conserved (46,47), regulation of longevity by miRNAs is expected in humans. Indeed, several human miRNAs target components of well-known conserved longevity pathways (32) including IGF (miR-1, miR-7, miR-122, miR-206 miR-320, and miR-375) (48,49,50) steroid (miR-122, miR-14, let-7) (32,51,52) and target of rapamycin (TOR) (miR-21) (53) signaling (FIG. 1). In addition, some of these miRNAs have been linked to human aging-related disorders such as heart (54-64), muscle (59), and neurodegenerative disease (65,66) (FIG. 1).

The present invention addresses the need for elucidating the role of miRNAs and their target genes in human longevity, and their impact on age-related diseases.

### SUMMARY OF THE INVENTION

A method is provided for determining if a subject is likely to develop an age-related disease comprising determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not likely to develop an age-related disease when the sample contains levels of the miRNAs above the respective predetermined control levels for each mRNA.

Also provided is a method for treating a subject for an age-related disease comprising determining if a subject is likely to develop an age-related disease comprising a) empirically determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not suitable for treatment when the sample contains levels of the miRNAs above the respective predetermined control levels for each mRNA, and as suitable for treatment when the sample contains levels of the miRNAs below the respective predetermined control levels for each mRNA, and b) administering to a subject who has been identified as suitable for treatment in a) a treatment for an age-related disease, so as to thereby treat the subject.

Also provided is a method for treating a subject for an age-related disease comprising administering to the subject an amount of an isolated miR-142, miR-101, miR-301b, miR148a, miR21, miR-29c, miR30e, miR27a and miR15a effective to treat an age-related disease in a subject.

Also provided is a method for reducing the risk that a subject will suffer an age-related disease comprising administering to the subject an amount of an isolated miR-142, miR-101, miR-301b, miR148a, miR21, miR-29c, miR30e, miR27a and miR15a effective to reduce the risk that a subject will suffer an age-related disease.

### BRIEF DESCRIPTION OF THE DRAWINGS

FIG. 1: MiRNAs involved in conserved pathways of longevity and their role in age-related diseases in humans.

FIG. 2. Steps involved in miRNA discovery using massively parallel sequencing and development of an automated analytical pipeline.

FIG. 3. Expression of miRNAs in (A) LCLs (Lymphoblastoid cell lines) that are significantly different between centenarians (n=20, mean age 101 yrs) and controls (n=20, mean age 74 yrs) with Fold Change >1.5 and FDR <0.05, and (2) plasma that are significantly different between centenarians (n=10, mean age 98.5 yrs) and controls (n=10, mean age 74.7 yrs) with Fold Change >5.0 and FDR <0.05.

FIG. 4A-C. Validation of longevity-associated miRNAs. Cross sectional analysis of miRNA expression patterns at different ages can differentiate whether a miRNA is A) age-related, B) longevity-associated with youthful preservation, C) Cross sectional expression patterns of hsa-miR-29c suggest the youthful preservation model.

FIG. 5. Average relative expression of miR-20a over 3 independent measurements by TaqMan qPCR in 2 centenarian LCLs. The lines are SD. CVs (mean/SD of 3 measurements) are indicated.

FIG. 6. IGF1 pathway subnetwork of longevity-associated miRNAs (red dots). Lines link miRNAs and their target (blue dots).

FIG. 7. IGF1R 3′ UTR targeted by multiple miRNAs.

FIG. 8A-B. Down-regulation of genes involved in IGF1 signaling (A) and significant reverse-correlations between these genes and longevity-associated miRNAs (B); centenarians: Red dots, controls: Blue dots

FIG. 9A-C. Network analyses. (A) Embedment of a group of functionally related genes in a base biological network. (B) Construction of the subnetwork as defined by the embedded genes and the underlying base network. Additional related genes are identified. (C) Identification of modules within the subnetwork. Modules are shown as groups of encircled green nodes.

FIG. 10. Luciferase 3′UTR reporter assays to determine molecular interactions between a miRNA and its target genes.

FIG. 11A-C. Downregulation of IGF1 gene expression (A) and AKT phosphorylation (B) in LCLs of centenarians harboring longevity-associated miRNA signature as compared to LCLs from centenarians without the signature. Reverse-correlation (C) of all individuals; centenarians (Red) and controls (Blue).

FIG. 12A-E. Effects of miR-142 overexpression on IIS and mTOR signaling in MCF7 cells. (A) Reduced IIS as measured by phosphorylation of IGF1R, AKT, and FOXO3 in response to IGF1 treatment. (B) Quantification of (A). (C) Reduced protein levels of INSR, IGF1R, and RICTOR. (D) Quantification of (C). (E) Reduced mRNA expression of INSR, PI3KR2, RICTOR, and mTOR by qPCR.

FIG. 13A-C. RICTOR is a direct target of miR-142. (A) No. of in silico predicted miR-142 targets. (B) 3′UTR reporter assays of RICTOR 3′UTR fragments. (C) Pull-down assay of Bi-miR-142.

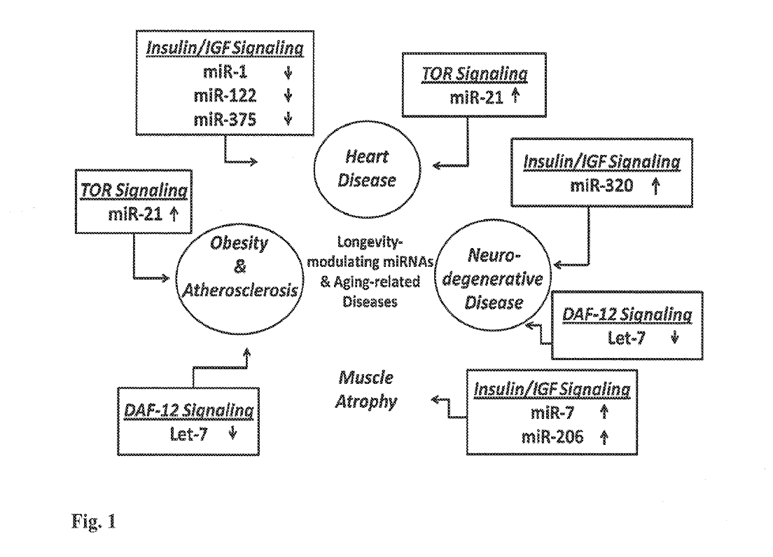

### DETAILED DESCRIPTION OF THE INVENTION

A method is provided for determining if a subject is likely to develop an age-related disease comprising determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not likely to develop an age-related disease when the sample contains levels of the miRNAs above the respective predetermined control levels for each mRNA.

Determining, as used herein, means experimentally determining, for example, using a machine or device, testing empirically.

Also provided is a method for treating a subject for an age-related disease comprising determining if a subject is likely to develop an age-related disease comprising a) empirically determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not suitable for treatment when the sample contains levels of the miRNAs above the respective predetermined control levels for each mRNA, and as suitable for treatment when the sample contains levels of the miRNAs below the respective predetermined control levels for each mRNA, and b) administering to a subject who has been identified as suitable for treatment in a) a treatment for an age-related disease, so as to thereby treat the subject.

In an embodiment of the methods, when the sample contains levels of the miRNAs below the predetermined control levels for each mRNA, the subject is identified as likely to develop an age-related disease.

In an embodiment of the methods, the sample comprises plasma or cell-free serum. In an embodiment of the methods, the sample comprises lymphoblastoid cells.

In an embodiment of the methods, a subject is identified as not likely to develop an age-related disease when all of miR-142, miR-101, miR-301b, miR148a, miR21, miR-29c, miR30e, miR27a and miR15a are at a level above their respective predetermined control levels.

In an embodiment of the methods, a subject is identified as likely to develop an age-related disease when all of miR-142, miR-101, miR-301b, miR148a, miR21, miR-29c, miR30e, miR27a and miR15a are at a level below their respective predetermined control levels.

In an embodiment of the methods, the method further comprises testing a sample from a subject identified as likely to develop an age-related disease with a test predictive of development of, or predisposition to type II diabetes, metabolic syndrome, a cardiovascular disease, hypertension, cognitive impairment, obesity, atherosclerosis, muscle atrophy or a neurodegenerative disease.

In an embodiment of the methods, the method further comprises treating a subject identified as likely to develop an age-related disease with a prophylactic treatment for an age-related disease.

In an embodiment of the methods, the method further comprises treating a subject identified as predisposed to, or likely to type II diabetes, metabolic syndrome, a cardiovascular disease, hypertension or cognitive impairment with a treatment for type II diabetes, metabolic syndrome, a cardiovascular disease, hypertension, cognitive impairment, obesity, atherosclerosis, muscle atrophy or a neurodegenerative disease, respectively.

In an embodiment of the methods, the age-related disease is type II diabetes, metabolic syndrome, a cardiovascular disease, hypertension or cognitive impairment. In an embodiment of the methods, the age-related disease is cardiovascular disease and is stroke, myocardial infarction, or a coronary vascular disease.

Hypertensive subjects, in an embodiment, are considered as those with self-reported pharmacological treatment or those who meet the criteria of The Seventh Report of the Joint National Committee on Prevention, Detection, Evaluation, and Treatment of High Blood Pressure, specifically, systolic blood pressure >140 mmHg or diastolic blood pressure >90 mmHg Type 2 diabetes mellitus (T2DM) in an embodiment is subjects on pharmacological treatment or using American Diabetes Association criteria of fasting glucose ≧126 mg/dl, and HbA1C>6.5%. Subjects with cardiovascular diseases, in an embodiment, are subjects with a history of acute non-fatal myocardial infarction, stroke and cardiac surgeries including angioplasty or coronary bypass surgery. Metabolic Syndrome subjects, in an embodiment, are subjects defined using the criteria of the National Cholesterol Education Program modified Adult Treatment Panel III Report, namely the presence of three or more of the following five attributes: waist circumference exceeding 102 cm (men) or 88 cm (women), triglycerides levels >150 mg/dl, HDL cholesterol <40 (men) or 50 (women), blood pressure ≧130/85, history of diabetes or glucose >100 mg/dl. Cognitive impairment (MCI/dementia) and test scores on neuropsychological tests are based on the Clinical Core procedures used in the Einstein Aging Study and overlaps substantially with the Uniform Data Set of the Alzheimer's Disease Centers. These neuropsychological tests are standardized, well-normed and divided into partially overlapping domains to establish clinical diagnoses.

Also provided is a method for treating a subject for an age-related disease comprising administering to the subject an amount of an isolated miR-142, miR-101, miR-301b, miR148a, miR21, miR-29c, miR30e, miR27a and miR15a effective to treat an age-related disease in a subject. Also provided is a method for reducing the risk that a subject will suffer an age-related disease comprising administering to the subject an amount of an isolated miR-142, miR-101, miR-301b, miR148a, miR21, miR-29c, miR30e, miR27a and miR15a effective to reduce the risk that a subject will suffer an age-related disease.

In an embodiment, the miR-142, miR-101, miR-301b, miR148a, miR21, miR-29c, miR30e, miR27a or miR15a is administered systemically. In an embodiment, the miR-142, miR-101, miR-301b, miR148a, miR21, miR-29c, miR30e, miR27a or miR15a is administered intravenously. In an embodiment, the miR-142, miR-101, miR-301b, miR148a, miR21, miR-29c, miR30e, miR27a or miR15a is administered in a pharmaceutically acceptable carrier. In an embodiment, the miR-142, miR-101, miR-301b, miR148a, miR21, miR-29c, miR30e, miR27a or miR15a administered is a locked nucleic acid miR-142, miR-101, miR-301b, miR148a, miR21, miR-29c, miR30e, miR27a or miR15a. A locked nucleic acid is a high-affinity RNA analog in which one or more of the ribose rings are “locked” in the ideal conformation for Watson-Crick binding. As a result, locked nucleic acid microRNAs exhibit high thermal stability when hybridized to a complementary DNA or RNA strand and also exhibit high stability in serum. In embodiments, the locked nucleic acid microRNA contains one, two or three modified ribose rings. Specifically, the ribose ring is connected by a methylene bridge between the 2′-O and 4′-C atoms. In an embodiment, the locked nucleic acid microRNA is administered with the sequence of a microRNA precursor. In an embodiment, the locked nucleic acid microRNA is administered with the sequence of a mature microRNA.

In an embodiment, the miR-142 is administered. In an embodiment, the amount of miR-142 administered is sufficient to decrease IGF1 signaling in a subject.

In an embodiment, the microRNA administered has the same sequence as a corresponding human microRNA. For example, the miR142 administered has the same sequence as a human miR142.

In an embodiment, the age-related disease is type II diabetes, metabolic syndrome, a cardiovascular disease, hypertension or cognitive impairment. In an embodiment, the age-related disease is cardiovascular disease and is stroke, myocardial infarction, or a coronary vascular disease.

In an embodiment of the methods described herein, the subject is a mammal. In a preferred embodiment, the subject is a human subject.

The methods described herein are useful in various settings. For example, in determining death risk of an individual in the case of a life insurance policy application, a determination that the individual shows the 9 miRNA signature at levels higher than control would suggest a good risk situation for the insurance carrier company.

In an embodiment, the miRNAs are of the miRNA precursor sequences as set forth in the Experimental Results section below. In an embodiment, the miRNA has a sequence as set forth in one of SEQ ID NOS:1-9. In an embodiment, the miRNA has a sequence as set forth in one of SEQ ID NO:1 or a mature form thereof.

As used herein, a predetermined control level is a value decided for a control system or entity. The concept of a control is well-established in the field, and can be determined, in a non-limiting example, empirically from non-afflicted subject(s) (versus afflicted subject(s)), such as an age-appropriate healthy subject. The predetermined control level and may be normalized as desired to negate the effect of one or more variables.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

The multitude of important roles played by miRNAs indicates that they are a critical genetic component of gene regulatory networks. However, quantification of miRNA has been technically challenging due to their small size, low copy number, interference from other small RNAs, and contamination by degradation products of mRNAs or other RNA species. Until recently, the only known and computationally predicted miRNAs have been interrogated using hybridization-based array methods, an assay of limited value due to cross-hybridization, array content, and the inability to discover novel miRNAs. Increased availability and affordability of massively parallel sequencing offer a dramatically improved method to gain a high-resolution view of miRNA expression (67). This technology has been utilized to quantify expression profiles of miRNAs in several species, including humans (68,69).

The discovery of miRNAs points to an entirely new regulatory module for control of biological processes. Increasingly, studies are linking altered miRNA function to disease mechanisms (70). It is hypothesized herein that miRNAs play a major role in modulating human lifespan and the aging process. This has been the case in some studies of model organisms (30,31,43). The important roles for miRNAs in human longevity disclosed herein provide a rational basis for intervention strategies using miRNA therapeutics that promote healthy aging. This is based on the fact that in contrast to other cellular mediators, miRNAs can be easily manipulated and therapies based on anti-miRs or miRNA mimics developed to repress pathological miRNAs (71,72) or overexpress protective miRNAs (38).

An innovative study design was effected involving a unique cohort of centenarians, their offspring, and age-matched, sex-matched controls without a family history of exceptional longevity, all of genetically homogeneous Ashkenazi Jewish (AJ) descent, collected by Dr. Nir Barzilai of Albert Einstein College of Medicine of Yeshiva University. The concept of miRNA regulation as a factor involved in extreme human longevity is novel. The determinations disclosed herein that there is a difference in miRNA expression levels in LCLs and plasma from centenarians as compared to controls opens up a new approach for studying modulation of longevity in humans. Also, the approach used herein for investigating the role of miRNAs in human longevity is novel. This combines an unbiased genome-wide discovery approach utilizing cutting-edge technologies for discovery of longevity-associated miRNAs and association/mechanistic studies using advanced methods to ascertain their functional relevance and biological significance.

The hypothesis that the maintenance of youthful miRNA expression patterns is beneficial and long-lived humans (e.g. centenarians) are enriched with “longevity-promoting” miRNAs that confer robustness to gene expression regulatory networks protecting against age-related deterioration was tested. Longevity in humans is an inherited trait. While the heritability of average life expectancy has been estimated to be only ˜25% (79,80), studies of centenarians indicate much stronger heritability at old age. For example, siblings of centenarians have a 4 times greater probability of surviving to age 90 than siblings of those with average life span (81). Living to age 100 is 17 and 8 times more likely for male or female siblings of centenarians, respectively, compared to their birth cohort (82). In addition, longevity is strongly inherited from parents whose age of death is over 70, and more so as age of parents' death increases, but not with parents who die before age 70 (81). These findings firmly established the utility of human centenarians as a model system to study the genetics of aging and longevity. Thus, genetic studies of centenarians are based on the premise that such research may help identify genetic factors that are either particularly enriched in these populations, due to positive effects on life span, or under-represented due to a negative impact on health. Indeed, centenarians show “positive phenotypes of aging”, including extended preservation of function, such as cognitive and vascular function, and resistance to age-related disease and frailty (73-78). Since the frequency of centenarians is only ˜1/10,000 individuals, the longevity factors may not be present in a younger (˜60-70 yrs) control population without a family history of longevity.

Study Population: AJ centenarians, their offspring, and controls. The genetically homogenous populations of Ashkenazi Jews (AJ) were studied and biological samples and phenotype data was collected from centenarians, their offspring and unrelated controls. The rationale of this study design is that if longevity is inherited, longevity-associated, measurable clinical and biological phenotypes can also be identified in the offspring of centenarians at an early age. Indeed, plasma high-density lipoprotein (HDL) cholesterol levels and lipoprotein particle sizes are dramatically higher in the offspring of centenarians (83,84) and are correlated with the cognitive function of centenarians (85). Several studies demonstrated that the offspring of centenarians have a markedly reduced prevalence of age-related diseases, such as cardiovascular disease, diabetes mellitus, and cancer, as compared to unrelated age-matched controls (76,86,87). These studies suggest that survival to exceptional old age may involve lower susceptibility to a broad range of age-related diseases, perhaps secondary to inhibition of basic mechanisms of aging. Thus, centenarian-enriched genotypes and molecular phenotypes such as gene expression levels in the offspring of centenarians suggest that this population can be used to test the heritability of exceptional longevity using age-matched controls. The genetic homogeneity of the AJ population contributes to the enhanced likelihood of successfully identifying genetic components of aging and longevity (88). The study population derives from The Longevity Genes Project (LGP). The subjects were already phenotyped with stored DNA and LCLs of AJ proband centenarians (n=542, >95), their offspring (offspring of parents with exceptional longevity, n=691, ages 60-85), and age- and gender-matched controls (offspring of parents with usual survival, n=601, ages 60-95).

LCLs for gene expression analysis. In this study, miRNAs with “general”, rather than tissue-specific, patterns of gene expression associated with human longevity were discovered as they are likely to be involved in “common” aging pathways (19). LCLs established from LGP subjects were studied because recent studies, including in this laboratory (1,2) have demonstrated that LCLs reflect functional characteristics of the donor and can be a useful tool for studying genotype-driven molecular endpoints such as gene expression, and expression quantitative trait locus (eQTL) analysis (3,4). Use of LCLs is justified because: 1) gene expression studies in various cell types, including LCLs, demonstrated that a large fraction of gene expression patterns are shared across different cell types (5); 2) LCLs act as surrogate tissues whenever there is correlation between the expression levels of LCLs and phenotypes of interest (6,7); 3) LCLs are an effective tool to identify disease genes by genome-wide eQTL analysis (8-15); and 4) there is increasing evidence that a large number of eQTLs originally identified in LCLs can also be detected in multiple primary tissues (16-18). Thus, studies in LCLs have been helpful for identifying functional regulatory variation and will be integral to improving understanding of genetics of gene expression in humans. Only positive results are interpreted, as in most large-scale discovery-based science (such as association studies). Expression profiling in LCLs provides a cost-efficient approach for identification of novel longevity-associated miRNAs, without the substantial cost, risk or inconvenience of collecting tissue from subjects (a logistically difficult task, unlikely to achieve adequate participation).

Plasma for miRNA analysis. Recent studies have revealed that miRNAs circulate in a cell-free form in blood (89,90) where they are relatively stable due to binding with other materials such as exosomes (91,90). Moreover, tissue miRNAs are released into circulating blood, serum or plasma. Such cell-free miRNAs can be studied as biomarkers for diverse diseases including cancers and cardiovascular disease (54, 90-97). MiRNA signatures in blood are similar in men and women (89), miRNA levels are similar in plasma and serum (91), and freeze/thaw as well as prolonged storage do not affect miRNA levels (91).

### EXPERIMENTAL RESULTS

### Example 1

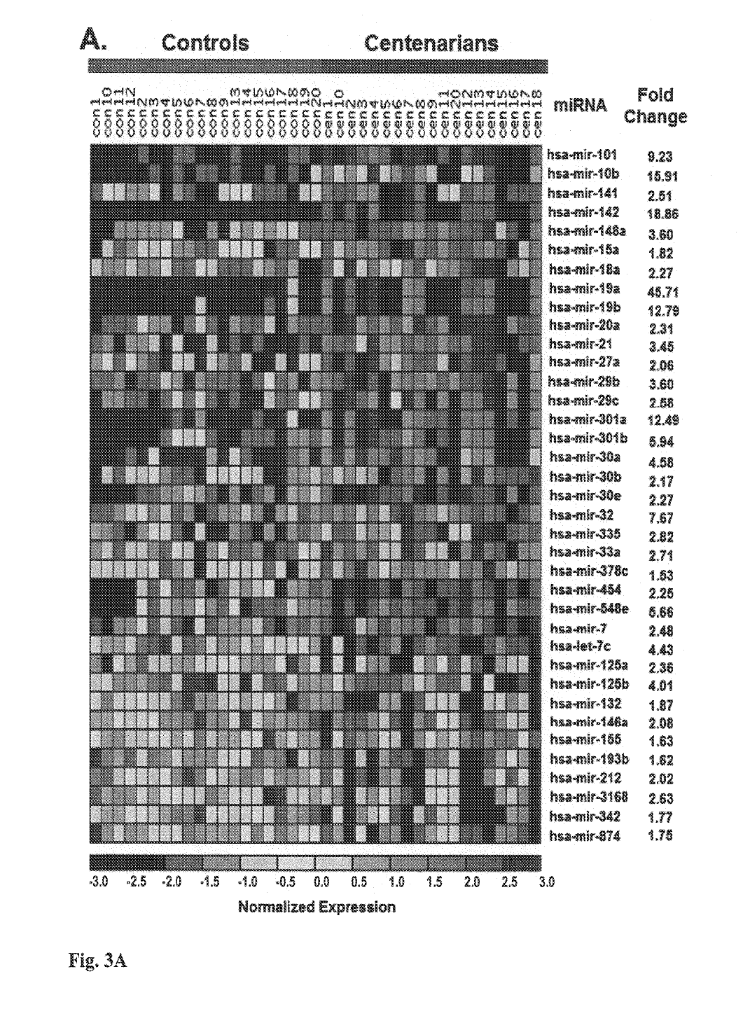

1) Discovery of miRNAs that are differentially expressed in LCLs of centenarians vs. controls. Preliminary work resulted in miRNA-seq and differential expression analysis of 3 centenarians (mean age 104) vs. 3 younger controls (mean age 63 controls). This was expanded to discover all possible miRNAs differentially expressed between 20 centenarians (mean age 101) and 20 controls (mean age 74 controls). 12-multiplex miRNA-seq was performed of individually barcoded libraries by Illumina Hi-Seq2000, which yielded a total of 2.7×10⁸ reads from centenarians and 3.1×10⁸ reads from controls. After removal of low quality reads and redundancy, there was a total of 1.1×10⁶ and 1.0×10⁶ unique reads for the centenarians and the controls, respectively. To analyze the computationally challenging miRNA-seq data, an automated analytical pipeline was developed (FIG. 2). Briefly, the sequencing data was provided from the Hi-Seq2000 sequencer in a standard fastq forma (98). Fastq files were trimmed of adapter sequences and low quality reads (more than 3 low quality base-calls), through a C++ program. These sequences were then collapsed to remove redundancy using the Galaxy Genome Browser tool fastx (99), followed by alignment to the known human miRNA/small RNA database or were put into the mirDeep pipeline for the discovery of novel miRNA (100). The miRNA tags matched were statistically normalized on a tags (determined miRNA) per total read (result from sequencer) basis (67). Following normalization, stringent criteria were applied for a miRNA to be considered for linear analysis, namely to be present in at least 50% (n=20) of the samples in greater than 10 copy numbers. After square-root transformation of data, a t-test was performed to generate nominal p-values (101). Correcting for multiple testing by a permutation procedure (102,103), miRNAs with a false discovery rate (FDR) <0.05 were considered significantly differentially expressed. A total of 37 miRNAs met this cutoff with a fold change >1.5, 28 of which had a fold change >2.0. Average read numbers for the 37 significant miRNAs ranged from 10 to over 480,000 (Table A1) with up to a 46-fold change. Of these 37 miRNAs, 26 have increased expression in centenarians. FIG. 4A is a heat map showing relative expression of the 37 significant miRNAs in controls and centenarians.

2) Cross platform comparison of differential miRNA expression. qRT-PCR analysis was conducted using TaqMan probes (Applied BioSystems) to compare the expression of differentially expressed miRNAs detected by Illumina sequencing (Appendix 1 and date not shown). While TagMan qPCR validated sequencing results, it clearly was less sensitive and specific than miRNA-seq in detecting relative expression and fold change. Nevertheless, qPCR method can reproducibly detect differential expression when read numbers of a miRNA are >100 and fold change >2.0.

3) Cross sectional analysis of miRNA expression in different age groups. Since the preliminary results were generated by differential analysis of two age groups, the mode of differential expression was determined. If up-regulation is simply age-related, expression will increase monotonically with age (FIG. 4A). In contrast, if up-regulation is longevity-related, patterns of youthful expression will be preserved (FIG. 4B). Such miRNA was found by a cross-sectional analysis in LCLs using TaqMan qPCR; the expression levels of miR-29c significantly decline with age (from 70s to early 90s) in control individuals while centenarians maintained the “highest” expression levels, suggesting that miR-29c is a longevity-associated miRNA (FIG. 4C).

4) Discovery of longevity-associated miRNA in plasma by TaqMan miRNA arrays. Recent studies have shown that circulating miRNAs can be profiled as biomarkers from small amounts of total RNA of serum or plasma using TaqMan qRT-PCR arrays (104-106). The TaqMan Human MicroRNA Array Panel A+B (Applied Biosystems), which detects 664 mature miRNAs and miRNAs* present in the Sanger mirBase, was used to profile miRNA expression in plasma samples of 10 centenarians (mean age 98.5) and 10 controls (mean age 74.7, controls). To isolate total RNA from plasma, the protocol by Mitchell et al. (91) was used with a slight modification using the mirVana PARIS kit (Roche). 200 μl of plasma as starting material, which provides a yield of >0.2 μg of small RNAs was used. Pre-amplification using the TaqMan PreAmp Mater Mix (Applied Biosystems) was performed to generate a miRNA cDNA library from each plasma sample, from which miRNA profiling was carried out (ABIPrism 7900HT). Data were analyzed with SDS Relative Quantification Software (v 2.3, Applied BioSystems). Mammalian U6 embedded in TaqMan Human MicroRNA Arrays was used as an endogenous control to normalize expression signaling. Relative expression levels of miRNAs were calculated using the comparative ΔΔCt method (107,108) followed by log 2-transformation. In order for a miRNA to be considered for differential analysis, it was required to be detected in at least 8 of the 20 samples. Fold changes in miRNAs were calculated by the equation 2-ΔΔCt. Statistical significance was determined using the Mann-Whitney test with multiple testing corrections by Benjamini-Hochberg method (109) to control for false discovery rate (FDR). MiRNAs with FDR <0.05 were considered significant. A total of 65 differentially expressed miRNAs with fold change >2.0 were discovered, among which 49 miRNAs show fold change >5.0 (FIG. 2B and Table 2). Interestingly, all these miRNAs have increased expression in centenarians.

MiRNA precursor sequences are set forth below:

hsa-mir-142 MI0000458
(SEQ ID NO: 1)
GACAGUGCAGUCACCCAUAAAGUAGAAAGCACUACUAACAGCACUGGA
GGGUGUAGUGUUUCCUACUUUAUGGAUGAGUGUACUGUG
hsa-mir-101-1 MI0000103
(SEQ ID NO: 2)
UGCCCUGGCUCAGUUAUCACAGUGCUGAUGCUGUCUAUUCUAAAGGUA
CAGUACUGUGAUAACUGAAGGAUGGCA
hsa-mir-301b MI0005568
(SEQ ID NO: 3)
GCCGCAGGUGCUCUGACGAGGUUGCACUACUGUGCUCUGAGAAGCAGU
GCAAUGAUAUUGUCAAAGCAUCUGGGACCA
hsa-mir-148a MI0000253
(SEQ ID NO: 4)
GAGGCAAAGUUCUGAGACACUCCGACUCUGAGUAUGAUAGAAGUCAGU
GCACUACAGAACUUUGUCUC
hsa-mir-21 MI0000077
(SEQ ID NO: 5)
UGUCGGGUAGCUUAUCAGACUGAUGUUGACUGUUGAAUCUCAUGGCAA
CACCAGUCGAUGGGCUGUCUGACA
hsa-mir-29c MI0000735
(SEQ ID NO: 6)
AUCUCUUACACAGGCUGACCGAUUUCUCCUGGUGUUCAGAGUCUGUUU
UUGUCUAGCACCAUUUGAAAUCGGUUAUGAUGUAGGGGGA
hsa-mir-30e MI0000749
(SEQ ID NO: 7)
GGGCAGUCUUUGCUACUGUAAACAUCCUUGACUGGAAGCUGUAAGGUG
UUCAGAGGAGCUUUCAGUCGGAUGUUUACAGCGGCAGGCUGCCA
hsa-mir-27a MI0000085
(SEQ ID NO: 8)
CUGAGGAGCAGGGCUUAGCUGCUUGUGAGCAGGGUCCACACCAAGUCG
UGUUCACAGUGGCUAAGUUCCGCCCCCCAG
hsa-mir-15a MI0000069
(SEQ ID NO: 9)
CCUUGGAGUAAAGUAGCAGCACAUAAUGGUUUGUGGAUUUUGAAAAGG
UGCAGGCCAUAUUGUGCUGCCUCAAAAAUACAAGG

### Materials and Methods.

1) Discovery of longevity-associated miRNA in LCLs by miRNA-seq and in plasma by TaqMan miRNA arrays. To discover miRNAs associated with longevity in humans, miRNA-seq by Illumina Hi-Seq2000 is employed to comprehensively analyze all possible miRNAs expressed in LCLs, and TaqMan miRNA arrays for plasma miRNAs. 80 individuals are selected from controls at different ages uniformly distributed from 60-90 and 20 centenarians (total 100) for discovery. The sample size gives reasonable statistical power to account for individual variation in expression levels (Table 1).

TABLE 1
Statistical power of monotonicity test.

| | Sig. | Difference between 90 yr | |
|---|---|---|---|
| Stage | Level | controls and centenarians | Power |
| Discovery | 0.05 | 0.75 SD | 0.80 |
| (n = 100) | | | |

2) Validation of longevity-associated miRNAs. The longevity-associated miRNAs are validated based on cross-sectional expression patterns. Since preliminary results indicated that significantly differentially expressed miRNAs are mostly upregulated in centenarians as compared to controls (FIG. 4), upregulation is used as a model. If up-regulation is simply age-related, expression will increase monotonically with age in all individuals (FIG. 4A). In contrast, if up-regulation is longevity-related, patterns of youthful expression will be preserved both in centenarians and offspring (FIG. 4B). Also considered is the presence of significantly down-regulated miRNAs in centenarians with youthful maintenance of expression patterns, namely increased expression with age in controls but low levels of expression in centenarians and offspring. TaqMan qPCR analysis of longevity-associated miRNAs discovered in LCLs is conducted using LCL samples from 500 centenarians, 500 offspring, and 500 controls at various ages. Similarly, TaqMan qPCR analysis of longevity-associated miRNAs discovered in plasma using plasma samples from 500 centenarians, 500 offspring, and 500 controls at various ages. As described previously, for plasma miRNAs TaqMan PreAmp Master Mix and miRNA assay kit is used with spiked-in synthetic C. elegans miRNAs a signal normalizer. Two-tailed two sample Student's t tests and ANOVA are used for statistical evaluation. The top 20 longevity-associated miRNAs discovered in LCLs and plasma are used for validation analysis, prioritized based on fold change, read numbers, biological relevance to aging and longevity according to their predicted and validated target genes as well as overlap between the LCLs and plasma results. The results based on comparison between centenarians and controls (age, 70s) indicate that a total of 9 miRNAs were up-regulated both in LCLs and plasma of centenarians compared to controls (Table 2), including the candidate longevity-associated miRNAs, miR-29c (FIG. 5C), and miR-101, miR-148a, and miR-27a, all of which were shown to be down-regulated with age in PBMCs (110).

TABLE 2
MiRNAs up-regulated both in LCLs and Plasma of centenarians
as compared to controls with fold change. FDR < 0.05

Fold Change

| miRNAs | LCLs | Plasma |
|---|---|---|
| hsa-miR-142 | 18.86 | 10.84 |
| hsa-miR-101 | 9.23 | 5.83 |
| hsa-miR-301b | 5.94 | 5.06 |
| hsa-miR-148a | 3.6 | 5.51 |
| hsa-miR-21 | 3.45 | 5.05 |
| hsa-miR-29c | 2.58 | 5.64 |
| hsa-miR-30e | 2.27 | 6.63 |
| hsa-miR-27a | 2.06 | 5.95 |
| hsa-miR-15a | 1.82 | 28.45 |

Data Analysis and Statistical Consideration. To analyze LCL miRNA-seq data, an automated analytical pipeline is used (FIG. 2). Expression profiling of each subject is normalized by its total number of reads. Square root transformation is applied to the normalized read count for regression analysis described below. To analyze plasma TaqMan miRNA array Data, SDS Relative Quantification Software (v2.3 Applied BioSystems) is used and signal normalization by U6. The relative expression levels of miRNAs is calculated using the comparative ΔΔCt method followed by log 2-transformation. To identify longevity-associated miRNAs, the test of monotonicity based on non-parametric regression is applied, as proposed by Bowman et al. (111) and implemented in R-package sm (112). For those miRNAs that show significant non-monotonicity, a linear regression model is further fitted for subjects younger than 95 years old, and a t-test performed comparing those older than 95 with those between 80 to 90. Those miRNAs that show statistically significant negative slope in the linear regression model and show higher expression among centenarians (age >95) compared to those between 80-90 are selected for validation analysis. Similarly, those that show significant positive slope in the linear regression model and lower expression among centenarians compared to those between 80-90 are also selected for validation. In the data on miR-29c (FIG. 4C), a 1.6 standard deviation difference was observed between the 80-90 year old control groups and centenarians.

The validation study using expression data from LCLs and plasma is conducted in two independent analyses. First, for validation, in combined samples of controls and centenarians, the same test for monotonicity is performed as described previously. Second, expression levels of controls are compared with those of offspring. Under the standard framework of a linear model, it is tested if the slope of controls is different from offspring under the constraints that they have the same miRNA levels at age 50-60 y. A miRNA is considered to be validated by cross-sectional data only if it shows statistical significance in both tests at significance of 0.05. Since the two tests are independent, the false positive rate for each miRNA is controlled at 0.052=0.0025, and is equivalent to control for overall Type-I error at 0.05 after Bonferroni correction, assuming 20 miRNAs are to be validated. In the test of monotonicity, based on simulation studies, it is estimated that if centenarians have the same miRNA expression levels as 65 years old controls, and at the age of 90, the controls are 0.3 standard deviations below the centenarians, an 85% power to detect this degree of non-monotonicity is available (Table 3).

TABLE 3
Statistical Power for Validation Studies

| | | Sig. | Detectable | |
|---|---|---|---|---|
| Analysis | Sample | Level | Difference | Power |
| 1. Monotonicity | 500 controls | 0.05 | 0.30 SD btw 90 | 0.85 |
| Test | and 500 Cent. | | yr controls and | |
| | (Plasma or LCLs) | | Cent. | |
| 2. Difference in | 500 controls vs. | 0.05 | 0.6(0.5) SD at 90 | 0.9(0.8) |
| Slopes | 500 offspring | | yr btw controls | |
| | (LCL/Plasma) | | and offspring | |

In the test of slope differences between offspring and controls, assuming the two groups have the same miRNA levels at age 65 but subsequently decline at different rates, a statistical power of 0.90 (and 0.80) is available to detect a difference in slopes that results in a 0.6 (and 0.5) standard deviation difference in mean miRNA levels at the age of 90 between the two groups (estimated using G*Power) (113). This cross-sectional analysis allows validation of longevity-associated miRNAs that show the maintenance of youthful expression patterns in centenarians and offspring. Estimates of “narrow sense’ heritability (h2) can be made from the slope of linear regression of each parent on the mean value of offspring (114,83,115).

Data Analysis and Statistical Consideration. For binary phenotypes, a logistic regression model adjusted for age, sex, education, and other confounders is used and for continuous phenotypes, regular linear regression adjusted for age, sex, education, and other confounders is used to detect association. One miRNA and one target mRNA are considered at a time. Based on a linear regression model, at a significance level of 0.00083 (Bonferroni correction, 0.05/60), a statistical power of 0.80 is available to detect miRNA/mRNA that explains 4% of total variation in phenotypes. Multiple miRNA/mRNAs are entertained in the regression model together using model selection methods. Exhaustive searches can be performed, or otherwise Bayesian variable selection methods (142) are used. Finally, miRNA and mRNA expression results are used together with clinical phenotypes, and lifespan for causal modeling (‘Mendelian Randomization’) studies (143).

To confirm the targets of longevity-associated miRNAs, a luciferase reporter assay has been established using pMIR-REPORT vector (Ambion). Using this system, the interaction between miR-493 and its predicted target, eEF1A 3′UTR, was validated. The putative 3′UTR target site downstream of a luciferase reporter gene was cloned (FIG. 10A) and HeLa cells cotransfected with this vector together with miR-493 or the scrambled negative control (Ambion). Normalized luciferase activity of HeLa cells transfected with miR-493 was significantly decreased as compared to negative control (p=0.003, t test, FIG. 10C). It was further tested whether the interaction between miR-493 and eEF1A1 3′UTR is direct or indirect by generating 2 mutations in the miR-493 predicted binding site in eEF1A1 3′UTR (FIG. 10B). Mutated eEF1A1 3′UTR was not regulated by miR-493 (FIG. 10C), demonstrating that eEF1A1 is a direct target of miR-493 through its binding to 3′UTR.

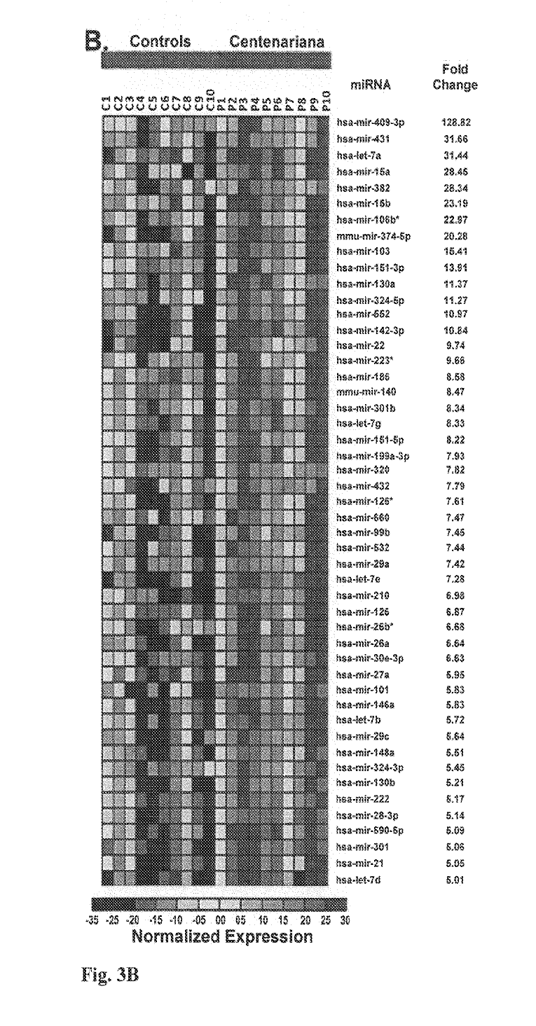

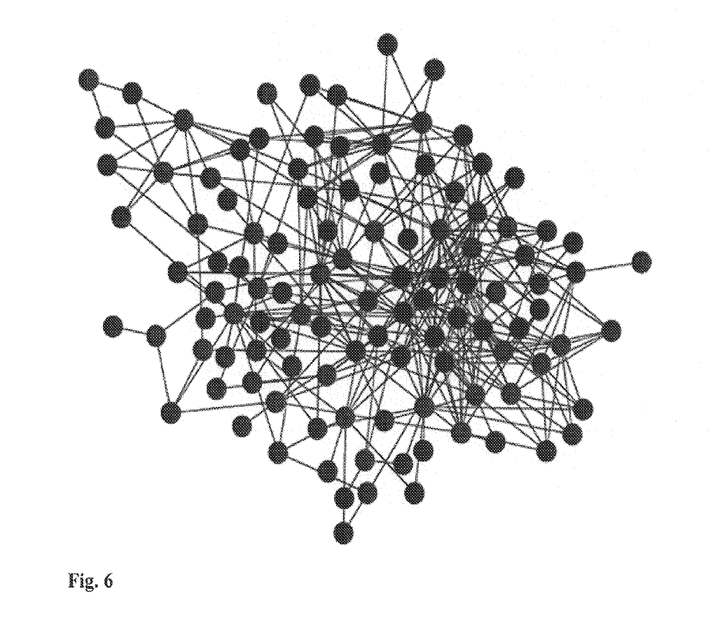

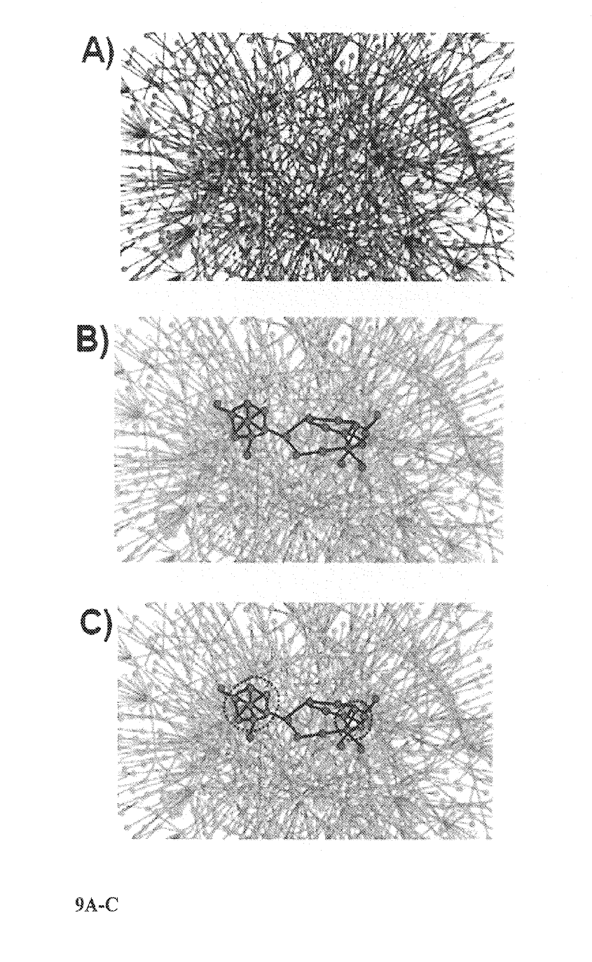

To identify all possible “real” targets, miRNA pull-down assay and CLIP technology (144,145) is used for 2-3 robust longevity-associated miRNAs. The causal relationship between longevity-associated miRNAs and reduced IGF1 signaling through down-regulation of key genes involved in this pathway can be determined utilizing established methods to measure IGF1-induced cell signaling, gene expression changes, cell cycle profiles, and stress resistance (1,2). Significant reverse correlations were found (FIGS. 11A & 11B) in both expression levels and IGF1 signaling as measured by AKT phosphorylation after IGF1 treatment (1,2) between IGF1 and longevity-associated miRNAs predicted to target this gene in LCLs from a subset of centenarians who harbor longevity-associated miRNA signature (FIG. 3). While the reverse correlations in expression levels between IGF1 and longevity-associated miRNAs, e.g. miR-30b (FIG. 11C), from LCLs of all individuals were not as obvious as compared to a subset of centenarians with the longevity miRNA signature. These results suggest that down-regulation of IGF1 signaling through gene regulation by miRNA may in part contribute to longevity for a subset of centenarians.

### Example 2

Functional role of longevity-associated miRNAs in modulation of conserved pathways of aging. MiRNAs alter cell and tissue phenotypes through alteration of target gene expression. To prioritize candidate miRNAs for comprehensive functional assays using an in vitro cell culture model, in silico prediction tools were used to identify targets genes and pathways of longevity-associated miRNAs as described (147). It was tested if target genes of longevity-associated miRNAs are part of known gene networks that impact on aging in general, using an online database and network analysis tool such as the NetAge database (148) and the Human Ageing Genomic Resources (HAGR) (149). Based on these predictions, a possible inverse-correlation was tested for in expression levels between a miRNA and its predicted target mRNAs by qPCR analysis measuring both “endogenous levels” in LCLs and regulated levels after overexpression using mimics or knock-down using anti-miRs. For example, target sites of the 41 differentially expressed miRNAs in LCLs showed overrepresentation of genes involved in the insulin/IGF-1 (IIS) signaling pathway, the first and best characterized conserved pathway of aging. Reduced-function or reduced-expression of the components in the IIS pathway universally extends life span and delay the onset and progression of aging-related diseases in animal models. Whether the longevity-associated miRNAs target the conserved IIS pathway as reported in C. elegans was tested (150).

A negative correlation was demonstrated between the upregulated miRNAs in centenarians and the expression of IIS pathway genes by qPCR analysis, and IIS signaling as measured by phospho-AKT, in LCLs. To establish causal relationships between longevity-associated miRNAs and IIS, 10 miRNAs found to be upregulated in “both LCLs and plasma” of centenarians were overexpressed using MCF7 cells and HepG2 cells. MiR-142, miR-29b, miR-29c reduced IIS gene expression and signaling in MCF7 cells, while miR-142, miR-19a, miR-101 did so in HepG2 cells. MiR-142 had the largest impact on IIS in both cell lines.

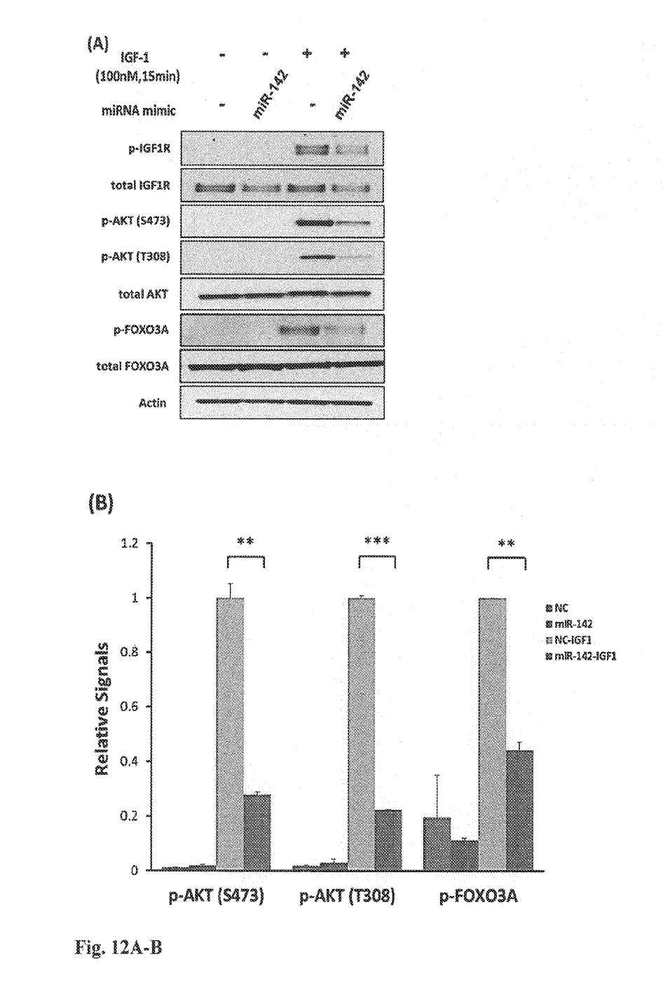

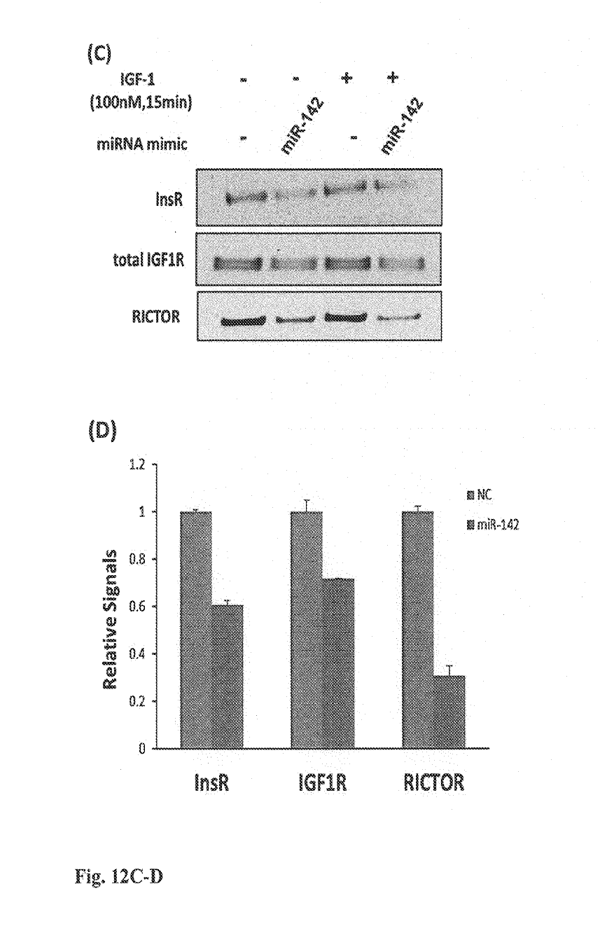

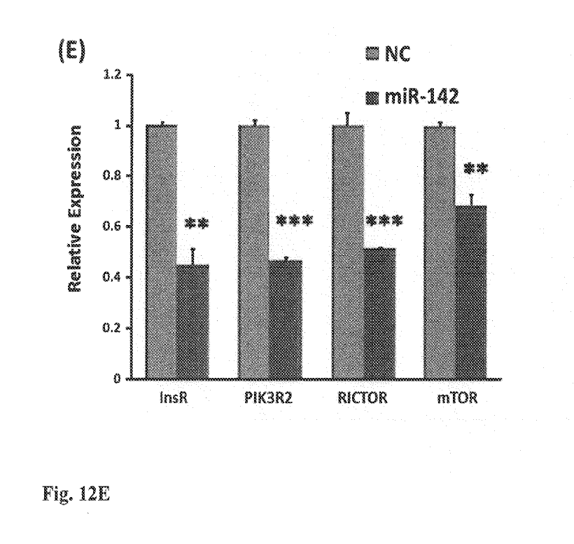

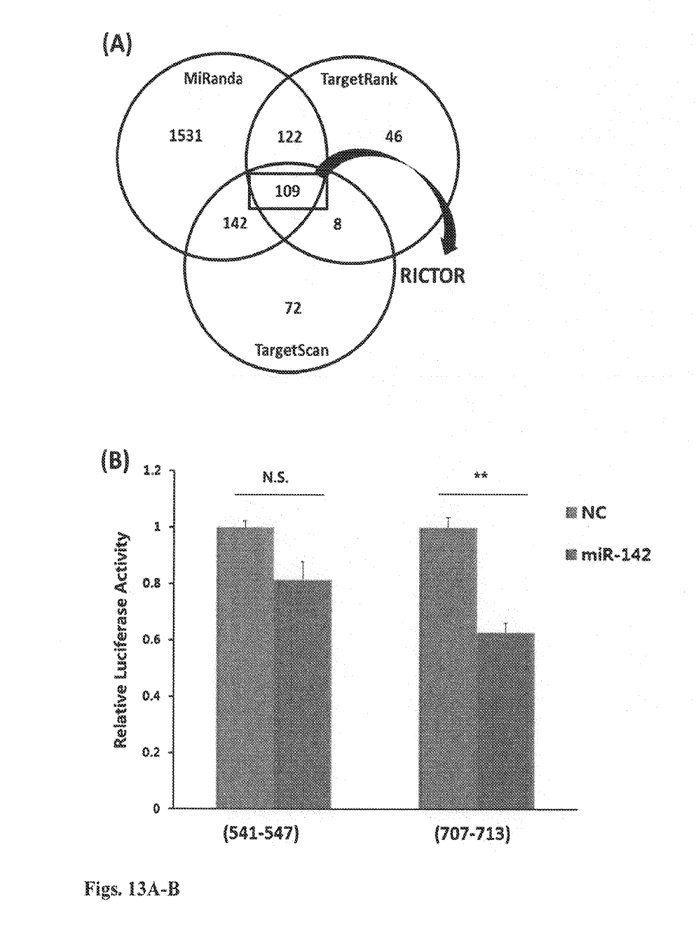

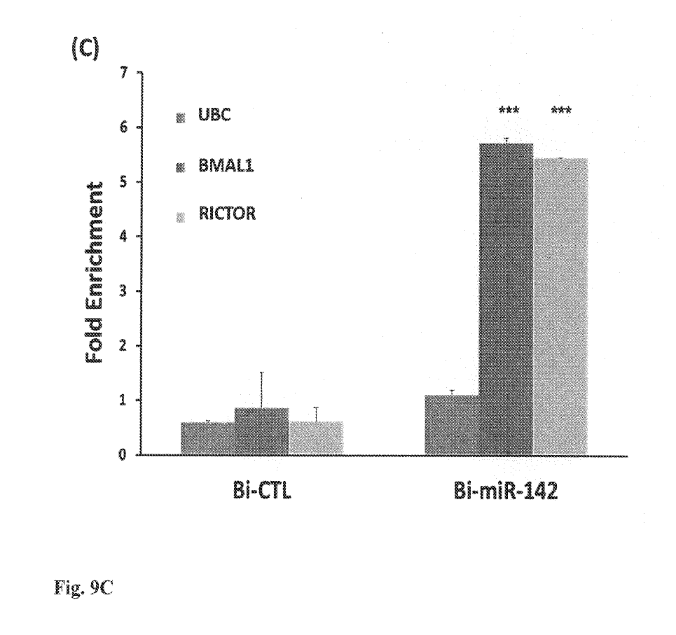

Overexpression of miR-142 reduced: i) protein levels of IGF1R, INSR, RICTOR; ii) AKT phosphorylation at both 5473 and T308 sites; iii) FOXO3 phosphorylation; and iv) mRNA levels of INSR, PI3KR2, RICTOR, and mTOR in MCF7 cells (FIG. 12). Luciferase reporter assays of fragments containing RICTOR 3′UTR predicted to bind miR-142 indicated that the second predicted site (707-713) is a likely target of miR-142 (FIGS. 13 A and B). These results suggest that down-regulation of IIS and mTOR signaling genes by miR-142 may in part contribute to longevity. To identify all possible mRNA targets of miR-142, a pull-down assay using Bi-miR-142 (3′-biotinylated-miR-142) was used and confirmed BMAL1 (151) and RICTOR as its direct target (FIG. 13 C).

### REFERENCES

1. Suh Y, Atzmon G, Cho M O, et al. Functionally significant insulin-like growth factor I receptor mutations in centenarians. Proc Natl Acad Sci USA 2008; 105:3438-42.
2. Tazearslan C, Huang J, Barzilai N, Suh Y. Impaired IGF1R signaling in cells expressing longevity-associated human IGF1R alleles. Aging Cell 2011; 10:551-4.
3. Cookson W, Liang L, Abecasis G, Moffatt M, Lathrop M. Mapping complex disease traits with global gene expression. Nat Rev Genet 2009; 10:184-94.
4. Montgomery S B, Dermitzakis E T. From expression QTLs to personalized transcriptomics. Nat Rev Genet 2011; 12:277-82.
5. Monks S A, Leonardson A, Zhu H, et al. Genetic inheritance of gene expression in human cell lines. Am J Hum Genet 2004; 75:1094-105.
6. Greenawalt D M, Dobrin R, Chudin E, et al. A survey of the genetics of stomach, liver, and adipose gene expression from a morbidly obese cohort. Genome Res 2011; 21:1008-16.
7. Mitchell G F, Verwoert G C, Tarasov K V, et al. Common Genetic Variation in the 3-BCL11B Gene Desert Is Associated with Carotid-Femoral Pulse Wave Velocity and Excess Cardiovascular Disease Risk: The AortaGen Consortium. Circulation Cardiovascular genetics 2011.
8. Moffatt M F, Kabesch M, Liang L, et al. Genetic variants regulating ORMDL3 expression contribute to the risk of childhood asthma. Nature 2007; 448:470-3.
9. Libioulle C, Louis E, Hansoul S, et al. Novel Crohn disease locus identified by genome-wide association maps to a gene desert on 5p13.1 and modulates expression of PTGER4. PLoS Genet 2007; 3:e58.
10. Lango Allen H, Estrada K, Lettre G, et al. Hundreds of variants clustered in genomic loci and biological pathways affect human height. Nature 2010; 467:832-8.
11. Soranzo N, Rivadeneira F, Chinappen-Horsley U, et al. Meta-analysis of genome-wide scans for human adult stature identifies novel Loci and associations with measures of skeletal frame size. PLoS Genet 2009; 5:e1000445.
12. Anttila V, Stefansson H, Kallela M, et al. Genome-wide association study of migraine implicates a common susceptibility variant on 8q22.1. Nat Genet 2010; 42:869-73.
13. Simon-Sanchez J, Schulte C, Bras J M, et al. Genome-wide association study reveals genetic risk underlying Parkinson's disease. Nat Genet 2009; 41:1308-12.
14. Dupuis J, Langenberg C, Prokopenko I, et al. New genetic loci implicated in fasting glucose homeostasis and their impact on type 2 diabetes risk. Nat Genet 2010; 42:105-16.
15. Voight B F, Scott U, Steinthorsdottir V, et al. Twelve type 2 diabetes susceptibility loci identified through large-scale association analysis. Nat Genet 2010; 42:579-89.
16. Bullaughey K, Chavarria C I, Coop G, Gilad Y. Expression quantitative trait loci detected in cell lines are often present in primary tissues. Hum Mol Genet 2009; 18:4296-303.
17. Heintzman N D, Hon G C, Hawkins R D, et al. Histone modifications at human enhancers reflect global cell-type-specific gene expression. Nature 2009; 459:108-12.
18. Hovatta I, Zapala M A, Broide R S, et al. DNA variation and brain region-specific expression profiles exhibit different relationships between inbred mouse strains: implications for eQTL mapping studies. Genome biology 2007; 8:R25.
19. Wheeler H E, Kim S K. Genetics and genomics of human ageing. Philosophical transactions of the Royal Society of London Series B, Biological sciences 2011; 366:43-50.
20. Long J M, Lahiri D K. MicroRNA-101 downregulates Alzheimer's amyloid-beta precursor protein levels in human cell cultures and is differentially expressed. Biochemical and biophysical research communications 2011; 404:889-95.
21. Vilardo E, Barbato C, Ciotti M, Cogoni C, Ruberti F. MicroRNA-101 regulates amyloid precursor protein expression in hippocampal neurons. The Journal of biological chemistry 2010; 285:18344-51.
22. Kriegel A J, Liu Y, Fang Y, Ding X, Liang M. The miR-29 Family: Genomics, Cell Biology, and Relevance to Renal and Cardiovascular Injury. Physiol Genomics 2012.
23. Park S Y, Lee J H, Ha M, Nam J W, Kim V N. miR-29 miRNAs activate p53 by targeting p85 alpha and CDC42. Nat Struct Mol Biol 2009; 16:23-9.
24. van Rooij E, Sutherland L B, Thatcher J E, et al. Dysregulation of microRNAs after myocardial infarction reveals a role of miR-29 in cardiac fibrosis. Proc Natl Acad Sci USA 2008; 105:13027-32.
25. Delay C, Mandemakers W, Hebert S S. MicroRNAs in Alzheimer's disease. Neurobiology of disease 2012.
26. Bahar R, Hartmann C H, Rodriguez K A, et al. Increased cell-to-cell variation in gene expression in ageing mouse heart. Nature 2006; 441:1011-4.
27. Somel M, Khaitovich P, Bahn S, Paabo S, Lachmann M. Gene expression becomes heterogeneous with age. Curr Biol 2006; 16:R359-60.
28. Southworth L K, Owen A B, Kim S K. Aging mice show a decreasing correlation of gene expression within genetic modules. PLoS Genet 2009; 5:e1000776.
29. Soltow Q A, Jones D P, Promislow D E. A network perspective on metabolism and aging. Integr Comp Biol 2010; 50:844-54.
30. Boehm M, Slack F. A developmental timing microRNA and its target regulate life span in C. elegans. Science 2005; 310:1954-7.
31. de Lencastre A, Pincus Z, Zhou K, Kato M, Lee S S, Slack F J. MicroRNAs Both Promote and Antagonize Longevity in C-elegans. Curr Biol 2010; 20:2159-68.
32. Ibanez-Ventoso C, Yang M, Guo S, Robins H, Padgett R W, Driscoll M. Modulated microRNA expression during adult lifespan in Caenorhabditis elegans. Aging Cell 2006; 5:235-46.
33. Lee R C, Feinbaum R L, Ambros V. The C. elegans heterochronic gene lin-4 encodes small RNAs with antisense complementarity to lin-14. Cell 1993; 75:843-54.
34. Bartel D P. MicroRNAs: genomics, biogenesis, mechanism, and function. Cell 2004; 116:281-97.
35. Filipowicz W, Bhattacharyya S N, Sonenberg N. Mechanisms of post-transcriptional regulation by microRNAs: are the answers in sight? Nat Rev Genet 2008; 9:102-14.
36. Brennecke J, Stark A, Russell R B, Cohen S M. Principles of microRNA-target recognition. PLoS Biol 2005; 3:e85.
37. Du T, Zamore P D. microPrimer: the biogenesis and function of microRNA. Development 2005; 132:4645-52.
38. Small E M, Olson E N. Pervasive roles of microRNAs in cardiovascular biology. Nature 2011; 469:336-42.
39. Saugstad J A. MicroRNAs as effectors of brain function with roles in ischemia and injury, neuroprotection, and neurodegeneration. J Cereb Blood Flow Metab 2010; 30:1564-76.
40. Griffiths-Jones S. The microRNA Registry. Nucleic Acids Res 2004; 32:D109-11.
41. Griffiths-Jones S, Saini H K, van Dongen S, Enright A J. miRBase: tools for microRNA genomics. Nucleic Acids Res 2008; 36:D154-8.
42. Lewis B P, Burge C B, Bartel D P. Conserved seed pairing, often flanked by adenosines, indicates that thousands of human genes are microRNA targets. Cell 2005; 120:15-20.
43. Pincus Z, Smith-Vikos, T., Slack, F. J. MicroRNA Predictors of Longevity in Caenorhabditis elegans. PLoS Genet 2011; 7:e1002306.
44. Ibanez-Ventoso C, Driscoll M. MicroRNAs in C. elegans Aging: Molecular Insurance for Robustness? Current genomics 2009; 10:144-53.
45. Bates D J, Li N, Liang R, et al. MicroRNA regulation in Ames dwarf mouse liver may contribute to delayed aging. Aging Cell 2010; 9:1-18.
46. Ibanez-Ventoso C, Vora M, Driscoll M. Sequence relationships among C. elegans, D. melanogaster and human microRNAs highlight the extensive conservation of microRNAs in biology. PLoS One 2008; 3:e2818.
47. Wang D E. MicroRNA Regulation and its Biological Significance in Personalized Medicine and Aging. Current genomics 2009; 10:143.
48. Shan Z X, Lin Q X, Fu Y H, et al. Upregulated expression of miR-1/miR-206 in a rat model of myocardial infarction. Biochemical and biophysical research communications 2009; 381:597-601.
49. Wang X H, Qian R Z, Zhang W, Chen S F, Jin H M, Hu R M. MicroRNA-320 expression in myocardial microvascular endothelial cells and its relationship with insulin-like growth factor-1 in type 2 diabetic rats. Clinical and experimental pharmacology & physiology 2009; 36:181-8.
50. Poy M N, Eliasson L, Krutzfeldt J, et al. A pancreatic islet-specific microRNA regulates insulin secretion. Nature 2004; 432:226-30.
51. Bethke A, Fielenbach N, Wang Z, Mangelsdorf D J, Antebi A. Nuclear hormone receptor regulation of microRNAs controls developmental progression. Science 2009; 324:95-8.
52. Broue F, Liere P, Kenyon C, Baulieu E E. A steroid hormone that extends the lifespan of Caenorhabditis elegans. Aging Cell 2007; 6:87-94.
53. Vinciguerra M, Sgroi A, Veyrat-Durebex C, Rubbia-Brandt L, Buhler L H, Foti M. Unsaturated fatty acids inhibit the expression of tumor suppressor phosphatase and tensin homolog (PTEN) via microRNA-21 up-regulation in hepatocytes. Hepatology 2009; 49:1176-84.
54. Cheng Y, Tan N, Yang J, et al. A translational study of circulating cell-free microRNA-1 in acute myocardial infarction. Clin Sci (Lond) 2010; 119:87-95.
55. D'Alessandra Y, Devanna P, Limana F, et al. Circulating microRNAs are new and sensitive biomarkers of myocardial infarction. European heart journal 2010 31:2765-73.
56. Han M, Toli J, Abdellatif M. MicroRNAs in the cardiovascular system. Current opinion in cardiology 2011; 26:181-9.
57. Cheng Y, Ji R, Yue J, et al. MicroRNAs are aberrantly expressed in hypertrophic heart: do they play a role in cardiac hypertrophy? The American journal of pathology 2007; 170:1831-40.
58. Cheng Y, Liu X, Zhang S, Lin Y, Yang J, Zhang C. MicroRNA-21 protects against the H(2)O(2)-induced injury on cardiac myocytes via its target gene PDCD4. Journal of molecular and cellular cardiology 2009; 47:5-14.
59. Hamrick M W, Herberg S, Arounleut P, et al. The adipokine leptin increases skeletal muscle mass and significantly alters skeletal muscle miRNA expression profile in aged mice. Biochemical and biophysical research communications 2010; 400:379-83.
60. Hulsmans M, De Keyzer D, Holvoet P. MicroRNAs regulating oxidative stress and inflammation in relation to obesity and atherosclerosis. Faseb J 2011.
61. Sayed D, He M, Hong C, et al. MicroRNA-21 is a downstream effector of AKT that mediates its antiapoptotic effects via suppression of Fas ligand. The Journal of biological chemistry 2010; 285:20281-90.
62. Sayed D, Hong C, Chen I Y, Lypowy J, Abdellatif M. MicroRNAs play an essential role in the development of cardiac hypertrophy. Circulation research 2007; 100:416-24.
63. Tatsuguchi M, Seok H Y, Callis T E, et al. Expression of microRNAs is dynamically regulated during cardiomyocyte hypertrophy. Journal of molecular and cellular cardiology 2007; 42:1137-41.
64. van Rooij E, Sutherland L B, Liu N, et al. A signature pattern of stress-responsive microRNAs that can evoke cardiac hypertrophy and heart failure. Proc Natl Acad Sci USA 2006; 103:18255-60.
65. Niwa R, Zhou F, Li C, Slack F J. The expression of the Alzheimer's amyloid precursor protein-like gene is regulated by developmental timing microRNAs and their targets in Caenorhabditis elegans. Developmental biology 2008; 315:418-25.
66. Provost P. MicroRNAs as a molecular basis for mental retardation, Alzheimer's and prion diseases. Brain research 2010; 1338:58-66.
67. Creighton C J, Reid J G, Gunaratne P H. Expression profiling of microRNAs by deep sequencing. Brief Bioinform 2009; 10:490-7.
68. Morin R D, O'Connor M D, Griffith M, et al. Application of massively parallel sequencing to microRNA profiling and discovery in human embryonic stem cells. Genome Res 2008; 18:610-21.
69. Bar M, Wyman S K, Fritz B R, et al. MicroRNA discovery and profiling in human embryonic stem cells by deep sequencing of small RNA libraries. Stem Cells 2008; 26:2496-505.
70. Vidal M, Cusick M E, Barabasi A L. Interactome networks and human disease. Cell 2011; 144:986-98.
71. Elmen J, Lindow M, Schutz S, et al. LNA-mediated microRNA silencing in non-human primates. Nature 2008; 452:896-9.
72. Lanford R E, Hildebrandt-Eriksen E S, Petri A, et al. Therapeutic silencing of microRNA-122 in primates with chronic hepatitis C virus infection. Science 2010; 327:198-201.
73. Perls T, Terry D. Understanding the determinants of exceptional longevity. Annals of Internal Medicine 2003; 139:445-9.
74. Perls T, Terry D. Genetics of exceptional longevity. Exp Gerontol 2003; 38:725-30.
75. Terry D F, Wilcox M, McCormick M A, Lawler E, Perls T T. Cardiovascular advantages among the offspring of centenarians. J Gerontol A Biol Sci Med Sci 2003; 58:M425-31.
76. Terry D F, McCormick M, Andersen S, et al. Cardiovascular disease delay in centenarian offspring: role of heat shock proteins. Ann N Y Acad Sci 2004; 1019:502-5.
77. Terry D F, Wilcox M A, McCormick M A, et al. Lower all-cause, cardiovascular, and cancer mortality in centenarians' offspring. J Am Geriatr Soc 2004; 52:2074-6.
78. Terry D F, Wilcox M A, McCormick M A, Perls T T. Cardiovascular disease delay in centenarian offspring. J Gerontol A Biol Sci Med Sci 2004; 59:385-9.
79. McGue M, Vaupel J W, Holm N, Harvald B. Longevity is moderately heritable in a sample of Danish twins born 1870-1880. J Gerontol 1993; 48:B237-44.
80. Herskind A M, McGue M, Holm N V, Sorensen T I, Harvald B, Vaupel J W. The heritability of human longevity: a population-based study of 2872 Danish twin pairs born 1870-1900. Hum Genet 1996; 97:319-23.
81. Perls T T, Bubrick E, Wager C G, Vijg J, Kruglyak L. Siblings of centenarians live longer. Lancet 1998; 351:1560.
82. Perls T, Kunkel L M, Puca A A. The genetics of exceptional human longevity. J Am Geriatr Soc 2002; 50:359-68.
83. Barzilai N, Atzmon G, Schechter C, et al. Unique lipoprotein phenotype and genotype associated with exceptional longevity. Jama 2003; 290:2030-40.
84. Barzilai N, Gabriely I, Gabriely M, Iankowitz N, Sorkin J D. Offspring of centenarians have a favorable lipid profile. Journal of the American Geriatrics Society 2001; 49:76-9.
85. Atzmon G, Gabriely I, Greiner W, Davidson D, Schechter C, Barzilai N. Plasma HDL levels highly correlate with cognitive function in exceptional longevity. J Gerontol A Biol Sci Med Sci 2002; 57:M712-5.
86. Andersen R V, Wittrup H H, Tybjaerg-Hansen A, Steffensen R, Schnohr P, Nordestgaard B G. Hepatic lipase mutations, elevated high-density lipoprotein cholesterol, and increased risk of ischemic heart disease: the Copenhagen City Heart Study. J Am Coll Cardiol 2003; 41:1972-82.
87. Atzmon G, Schechter C, Greiner W, Davidson D, Rennert G, Barzilai N. Clinical phenotype of families with longevity. Journal of the American Geriatrics Society 2004; 52:274-7.
88. Hirschhorn J N, Daly, M. J. Genome-wide association studies for common diseases and complex traits. Nat Rev Genet 2005; 6:95-108.
89. Hunter M P, Ismail N, Zhang X, et al. Detection of microRNA expression in human peripheral blood microvesicles. PLoS One 2008; 3:e3694.
90. Cortez M A, Calin G A. MicroRNA identification in plasma and serum: a new tool to diagnose and monitor diseases. Expert Opin Biol Ther 2009; 9:703-11.
91. Mitchell P S, Parkin R K, Kroh E M, et al. Circulating microRNAs as stable blood-based markers for cancer detection. Proc Natl Acad Sci USA 2008; 105:10513-8.
92. Chen J F, Murchison E P, Tang R, et al. Targeted deletion of Dicer in the heart leads to dilated cardiomyopathy and heart failure. Proc Natl Acad Sci USA 2008; 105:2111-6.
93. Dong S, Cheng Y, Yang J, et al. MicroRNA expression signature and the role of microRNA-21 in the early phase of acute myocardial infarction. The Journal of biological chemistry 2009; 284:29514-25.
94. Tanaka M, Oikawa K, Takanashi M, et al. Down-regulation of miR-92 in human plasma is a novel marker for acute leukemia patients. PLoS One 2009; 4:e5532.
95. Voellenkle C, van Rooij J, Cappuzzello C, et al. MicroRNA signatures in peripheral blood mononuclear cells of chronic heart failure patients. Physiol Genomics 2010; 42:420-6.
96. Fichtlscherer S, De Rosa S, Fox H, et al. Circulating microRNAs in patients with coronary artery disease. Circulation research 2010; 107:677-84.
97. Ji X, Takahashi R, Hiura Y, Hirokawa G, Fukushima Y, Iwai N. Plasma miR-208 as a biomarker of myocardial injury. Clin Chem 2009; 55:1944-9.
98. Cock P J, Fields C J, Goto N, Heuer M L, Rice P M. The Sanger FASTQ file format for sequences with quality scores, and the Solexa/Illumina FASTQ variants. Nucleic Acids Res 2009.
99. Blankenberg D, Von Kuster G, Coraor N, et al. Galaxy: a web-based genome analysis tool for experimentalists. Curr Protoc Mol Biol 2010; Chapter 19:Unit 19 0 1-21.
100. Friedlander M R, Chen W, Adamidi C, et al. Discovering microRNAs from deep sequencing data using miRDeep. Nat Biotechnol 2008; 26:407-15.
101. Robinson M D, Smyth G K. Moderated statistical tests for assessing differences in tag abundance. Bioinformatics 2007; 23:2881-7.
102. Dudoit S, Gilbert H N, van der Laan M J. Resampling-based empirical Bayes multiple testing procedures for controlling generalized tail probability and expected value error rates: focus on the false discovery rate and simulation study. Biometrical journal 2008; 50:716-44.
103. Storey J D, Tibshirani R. Statistical significance for genomewide studies. Proc Natl Acad Sci USA 2003; 100:9440-5.
104. Cui L, Qi Y, Li H, et al. Serum microRNA expression profile distinguishes enterovirus 71 and coxsackievirus 16 infections in patients with hand-foot-and-mouth disease. PLoS One 2011; 6:e27071.
105. Gui J, Tian Y, Wen X, et al. Serum microRNA characterization identifies miR-885-5p as a potential marker for detecting liver pathologies. Clin Sci (Lond) 2011; 120:183-93.
106. Wulfken L M, Moritz R, Ohlmann C, et al. MicroRNAs in renal cell carcinoma: diagnostic implications of serum miR-1233 levels. PLoS One 2011; 6:e25787.
107. Livak K J, Schmittgen T D. Analysis of relative gene expression data using real-time quantitative PCR and the 2(-Delta Delta C(T)) Method. Methods (San Diego, Calif. 2001; 25:402-8.
108. Schmittgen T D, Livak K J. Analyzing real-time PCR data by the comparative C(T) method. Nature protocols 2008; 3:1101-8.
109. Benjamini YaH, Y. Controlling the false discovery rate: a practical and powerful approach to multiple testing. J Roy Statist Soc Ser B 1995; 57:289-300.
110. Noren Hooten N, Abdelmohsen K, Gorospe M, Ejiogu N, Zonderman A B, Evans M K. microRNA expression patterns reveal differential expression of target genes with age. PLoS ONE 2010; 5:e10724.
111. Bowman A W, Jones, M. C. and Gijbels, I. Testing monotonicity of regression. JCompGraphStat 1998; 7:489-500.
112. Bowman AWaA, A. Computational aspects of nonparametric smoothing with illustrations from the sm library. Computational Statistics and Data Analysis 2003; 42: 545-60.
113. Faul F, Erdfelder, E., Lang, A. G., & Buchner, A. G*Power 3: A flexible statistical power analysis program for the social, behavioral, and biomedical sciences. Behavior Research Methods 2007; 39:175-91.
114. Falconer D, Mackay, T F C. Introduction to Quantitative Genetics. Essex, U K: Addison Wesley Longman; 1996.
115. Atzmon G, Barzilai N, Surks M I, Gabriely I. Genetic predisposition to elevated serum thyrotropin is associated with exceptional longevity. J Clin Endocrinol Metab 2009; 94:4768-75.
116. Cheung V G, Spielman R S. Genetics of human gene expression: mapping DNA variants that influence gene expression. Nat Rev Genet 2009; 10:595-604.
117. Redon R, Ishikawa S, Fitch K R, et al. Global variation in copy number in the human genome. Nature 2006; 444:444-54.
118. Hannula K, Lipsanen-Nyman M, Scherer S W, Holmberg C, Hoglund P, Kere J. Maternal and paternal chromosomes 7 show differential methylation of many genes in lymphoblast DNA. Genomics 2001; 73:1-9.
119. Akey J M, Biswas S, Leek J T, Storey J D. On the design and analysis of gene expression studies in human populations. Nature genetics 2007; 39:807-8; author reply 8-9.
120. Choy E, Yelensky R, Bonakdar S, et al. Genetic analysis of human traits in vitro: drug response and gene expression in lymphoblastoid cell lines. PLoS genetics 2008; 4:e1000287.
121. Carter K L, Cahir-McFarland E, Kieff E. Epstein-barr virus-induced changes in B-lymphocyte gene expression. Journal of virology 2002; 76:10427-36.
122. Arnone M I, Davidson E H. The hardwiring of development: organization and function of genomic regulatory systems. Development 1997; 124:1851-64.
123. Wray G A. The evolutionary significance of cis-regulatory mutations. Nat Rev Genet 2007; 8:206-16.
124. Brosh R, Shalgi R, Liran A, et al. p53-Repressed miRNAs are involved with E2F in a feed-forward loop promoting proliferation. Molecular systems biology 2008; 4:229.
125. Varambally S, Cao Q, Mani R S, et al. Genomic loss of microRNA-101 leads to overexpression of histone methyltransferase EZH2 in cancer. Science 2008; 322:1695-9.
126. Kozomara A, Griffiths-Jones S. miRBase: integrating microRNA annotation and deep-sequencing data. Nucleic Acids Res 2011; 39:D152-7.
127. Loscalzo J, Barabasi A L. Systems biology and the future of medicine. Wiley interdisciplinary reviews Systems biology and medicine 2011.
128. Barabasi A L, Gulbahce N, Loscalzo J. Network medicine: a network-based approach to human disease. Nat Rev Genet 2011; 12:56-68.
129. Grimson A, Farh K K, Johnston W K, Garrett-Engele P, Lim L P, Bartel D P. MicroRNA targeting specificity in mammals: determinants beyond seed pairing. Mol Cell 2007; 27:91-105.
130. John B, Enright A J, Aravin A, Tuschl T, Sander C, Marks D S. Human microRNA targets (vol 2, pg 1862, 2005). Plos Biology 2005; 3:1328-.
131. Krek A, Grun D, Poy M N, et al. Combinatorial microRNA target predictions. Nature Genetics 2005; 37:495-500.
132. Kuchenbauer F, Morin R D, Argiropoulos B, et al. In-depth characterization of the microRNA transcriptome in a leukemia progression model. Genome Res 2008; 18:1787-97.
133. Long D, Lee R, Williams P, Chan C Y, Ambros V, Ding Y. Potent effect of target structure on microRNA function. Nat Struct Mol Biol 2007; 14:287-94.
134. Wu S, Huang S, Ding J, et al. Multiple microRNAs modulate p21Cip1/Waf1 expression by directly targeting its 3′ untranslated region. Oncogene 2010.
135. Beissbarth T, Speed T P. GOstat: find statistically overrepresented Gene Ontologies within a group of genes. Bioinformatics 2004; 20:1464-5.
136. Tacutu R, Budovsky A, Fraifeld V E. The NetAge database: a compendium of networks for longevity, age-related diseases and associated processes. Biogerontology 2010; 11:513-22.
137. de Magalhaes J P, Budovsky A, Lehmann G, et al. The Human Ageing Genomic Resources: online databases and tools for biogerontologists. Aging Cell 2009; 8:65-72.
138. Chartrand G. Introductory Graph Theory. New York: Courier Dover Publications; 1985.
139. Girvan M, Newman M E. Community structure in social and biological networks. Proc Natl Acad Sci USA 2002; 99:7821-6.
140. Liu H, Brannon A R, Reddy A R, et al. Identifying mRNA targets of microRNA dysregulated in cancer: with application to clear cell Renal Cell Carcinoma. BMC systems biology 2010; 4:51.
141. Nicolae D L, Gamazon E, Zhang W, Duan S, Dolan M E, Cox N J. Trait-associated SNPs are more likely to be eQTLs: annotation to enhance discovery from GWAS. PLoS genetics 2010; 6:e1000888.
142. Chipman H, George, E., McCulloch, R. The Practical Implementation of Bayesian Model Selection. In: P. Lahiri, ed. Model Selection; 2001:65-134.
143. Davey Smith G, Ebrahim S ‘Mendelian randomization’: can genetic epidemiology contribute to understanding environmental determinants of disease? Int J Epidemiol 2003; 32:1-22.
144. Ule J, Jensen K, Mele A, Darnell R B. CLIP: a method for identifying protein-RNA interaction sites in living cells. Methods (San Diego, Calif. 2005; 37:376-86.
145. Chi S W, Zang J B, Mele A, Darnell R B. Argonaute HITS-CLIP decodes microRNA-mRNA interaction maps. Nature 2009; 460:479-86.
146. Yang L, Lin C, Liu W, et al. ncRNA- and Pc2 methylation-dependent gene relocation between nuclear structures mediates gene activation programs. Cell 2011; 147:773-88.
147. Gombar, S. et al. Comprehensive microRNA profiling in B-cells of human centenarians by massively parallel sequencing. BMC genomics 13, 353, doi:10.1186/1471-2164-13-353 (2012).
148. Tacutu, R., Budovsky, A. & Fraifeld, V. E. The NetAge database: a compendium of networks for longevity, age-related diseases and associated processes. Biogerontology 11, 513-522, doi:10.1007/s10522-010-9265-8 (2010).
149. de Magalhaes, J. P. et al. The Human Ageing Genomic Resources: online databases and tools for biogerontologists. Aging cell 8, 65-72, doi:ACE442 [pii]10.1111/j.1474-9726.2008.00442.x (2009).
150. de Lencastre, A. et al. MicroRNAs Both Promote and Antagonize Longevity in C-elegans. Curr Biol 20, 2159-2168 (2010).
151. Tan, X. et al. Clock-controlled mir-142-3p can target its activator, Bmal1. BMC molecular biology 13, 27, doi:10.1186/1471-2199-13-27 (2012).
152. Bokov, A. F. et al. Does reduced IGF-1R signaling in Igflr+/− mice alter aging? PLoS One 6, e26891, doi:10.1371/journal.pone.0026891 (2011).

1. A method for determining if a subject is likely to develop an age-related disease comprising determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not likely to develop an age-related disease when the sample contains levels of the miRNAs above the respective predetermined control levels for each mRNA.

2. A method for treating a subject for an age-related disease comprising determining if a subject is likely to develop an age-related disease comprising a) empirically determining the level of one or more of the following miRNAs in a sample obtained from the subject: miR-142, miR-101, miR-301b, miR148a, miR21, miR-29c, miR30e, miR27a and miR15a, and then comparing the levels of the miRNAs to predetermined control levels for each mRNA respectively, and identifying a subject as not suitable for treatment when the sample contains levels of the miRNAs above the respective predetermined control levels for each mRNA, and as suitable for treatment when the sample contains levels of the miRNAs below the respective predetermined control levels for each mRNA, and b) administering to a subject who has been identified as suitable for treatment in a) a treatment for an age-related disease, so as to thereby treat the subject.

3. The method of claim 1, wherein when the sample contains levels of the miRNAs below the predetermined control levels for each mRNA, the subject is identified as likely to develop an age-related disease.

4. The method of claim 1, wherein the sample comprises plasma or cell-free serum.

5. The method of claim 1, wherein the sample comprises lymphoblastoid cells.

6. The method of claim 1, wherein a subject is identified as not likely to develop an age-related disease when all of miR-142, miR-101, miR-301b, miR148a, miR21, miR-29c, miR30e, miR27a and miR15a are at a level above their respective predetermined control levels.

7. The method of claim 1, wherein a subject is identified as likely to develop an age-related disease when all of miR-142, miR-101, miR-301b, miR148a, miR21, miR-29c, miR30e, miR27a and miR15a are at a level below their respective predetermined control levels.

8. The method of claim 1, further comprising testing a sample from a subject identified as likely to develop an age-related disease with a test predictive of development of, or predisposition to type II diabetes, metabolic syndrome, a cardiovascular disease, hypertension, cognitive impairment, obesity, atherosclerosis, muscle atrophy or a neurodegenerative disease.

9. The method of claim 1, further comprising treating a subject identified as likely to develop an age-related disease with a prophylactic treatment for an age-related disease.

10. (canceled)

11. A method for treating a subject for an age-related disease comprising administering to the subject an amount of an isolated miR-142, miR-101, miR-301b, miR148a, miR21, miR-29c, miR30e, miR27a and miR15a effective to treat an age-related disease in a subject.

12. A method for reducing the risk that a subject will suffer an age-related disease comprising administering to the subject an amount of an isolated miR-142, miR-101, miR-301b, miR148a, miR21, miR-29c, miR30e, miR27a and miR15a effective to reduce the risk that a subject will suffer an age-related disease.

13. The method of claim 11, wherein the miR-142, miR-101, miR-301b, miR148a, miR21, miR-29c, miR30e, miR27a or miR15a is administered systemically.

14. The method of claim 11, wherein the miR-142, miR-101, miR-301b, miR148a, miR21, miR-29c, miR30e, miR27a or miR15a is administered intravenously.

15. The method of claim 11, wherein the miR-142, miR-101, miR-301b, miR148a, miR21, miR-29c, miR30e, miR27a or miR15a administered is a locked nucleic acid miR-142, miR-101, miR-301b, miR148a, miR21, miR-29c, miR30e, miR27a or miR15a.

16. The method of claim 11, wherein the miR-142 is administered.

17. The method of claim 11, wherein the microRNA administered has the same sequence as a corresponding human microRNA.

18. The method of claim 11, wherein the age-related disease is type II diabetes, metabolic syndrome, a cardiovascular disease, hypertension or cognitive impairment.

19. The method of claim 11, wherein the age-related disease is cardiovascular disease and is stroke, myocardial infarction, or a coronary vascular disease.

20. The method of claim 11, wherein the subject is a human subject.

21. The method of claim 16, wherein the amount of miR-142 administered is sufficient to decrease IGF1 signaling in a subject.
